(19) United States
(12) Patent Application Publication
(10) Pub. No.: US 2016/0009624 A1
(43) Pub. Date: Jan. 14, 2016

(54) THERMAL SALT-SPLITTING OF AMMONIUM PROPIONATE

(71) Applicant: DOW GLOBAL TECHNOLOGIES LLC, Midland, MI (US)

(72) Inventors: Sanjib Biswas, Freeport, TX (US); Barry B. Fish, Freeport, TX (US); Viet Pham, Freeport, TX (US); Binghe Gu, Midland, MI (US); Brandon A. Rodriguez, Freeport, TX (US)

(73) Assignee: Dow Global Technologies LLC, Midland, MI (US)

(21) Appl. No.: 14/764,841

(22) PCT Filed: Feb. 17, 2014
(86) PCT No.: PCT/US2014/016692

§ 371 Date: Jul. 30, 2015

Related U.S. Application Data
(60) Provisional application No. 61/794,573, filed on Mar. 15, 2013.

(51) Int. Cl. C07C 51/347

(52) U.S. Cl. CPC C07C 51/347

(57) ABSTRACT

A salt-splitting liquid and a process that uses the salt-splitting liquid to “split” ammonium propionate salts into ammonia (or amines) and propionic acid that minimizes increases in the viscosity.

### CROSS-REFERENCE TO RELATED APPLICATIONS

This application claims priority from provisional application Ser. No. 61/794,573, filed Mar. 15, 2013, which is incorporated herein by reference in its entirety.

### TECHNICAL FIELD

The present disclosure relates to a process for converting ammonium propionate to propionic acid.

### BACKGROUND

One bio-based process of producing propionic acid involves fermenting sugar(s) at or near neutral pH. Keeping the fermentation at or near neutral pH, however, leads to formation of a salt of propionic acid, most commonly ammonium propionate (AP). In order to arrive at the propionic acid, the AP salt from the fermentation process needs to be “split” into ammonia and propionic acid. Once split, the ammonia is recycled and the propionic acid can be converted to a more useful product.

There are, however, problems in “splitting” the AP salt into ammonia and propionic acid. For example, in one approach to “splitting” the AP salt an aqueous solution of AP is heated at either atmospheric or reduced pressure in a process called “thermal salt-splitting” (TSS). TSS removes water from the aqueous solution, which is undesirable as the viscosity of the resulting solution increases to the point where it is very difficult to handle.

There is a need, therefore, for a process of converting ammonium propionate in an aqueous mixture to propionic acid, which process minimizes viscosity increases by using a salt-splitting liquid to “split” the AP salt into ammonia and propionic acid.

### SUMMARY

The process of the invention is such a process of converting ammonium propionate in an aqueous mixture to propionic acid, the process comprising:

admixing a polar aprotic organic solvent and the aqueous mixture, where the ammonium propionate and the propionic acid are at least partially soluble in the polar aprotic organic solvent, to form a salt-splitting liquid;

heating the salt-splitting liquid to convert the ammonium propionate to propionic acid and ammonia, where heating the salt-splitting liquid produces a vapor phase containing at least water, ammonia and the solvent;

removing at least a portion of the water and the ammonia from the vapor phase during the heating; and

returning at least a portion of the solvent from the vapor phase back to the salt-splitting liquid.

Heating the salt-splitting liquid can be to a temperature of 100° C. to 130° C. The polar aprotic organic solvent is selected from the group consisting of C₄₋₈ alkanols, dimethylformamide (DMF), dimethylacetamide (DMA), N-methyl-2-pyrrolidone (NMP), dimethyl sulfoxide (DMSO), dimethylsulfone, dioxane, diglyme, or a combination thereof. The polar aprotic organic solvent is admixed with the aqueous mixture so that a weight ratio of propionic acid to polar aprotic organic solvent in the aqueous mixture is 0.6:1 or lower, preferably 0.18:1 to 0.58:1, more preferably 0.24:1 to 0.42:1.

Surprisingly, the process of the invention not only maintains low viscosity but also minimizes the formation of highly undesirable by-product amides. The loss of propionic acid in the overhead is also reduced.

### DETAILED DESCRIPTION

Fermentation broths, or aqueous mixtures, used in the production of propionic acid operate at or near neutral pH. In maintaining this neutral pH, salts of the propionic acid are produced, most commonly AP. To obtain the propionic acid, ammonia is “split” from the ammonium propionate.

A direct method of “splitting” the AP, into ammonia and propionic acid is to heat the aqueous mixture in a “thermal salt-splitting” or “TSS” process. During the heating, the ammonium propionate converts to propionic acid as ammonia and water are removed. The heating process also causes an increase in both the acidity of the aqueous mixture (e.g., the aqueous mixture becoming more acidic) and the concentration of propionic acid in the aqueous mixture as ammonia and water are removed.

In DE 2718363 it was suggested that the heating process can be used with ammonium salts of carboxylic acids (e.g., isobutyrate, acetate, adipate, (meth)acrylate, benzoate, and terephthalate) in water-soluble organic solvents (e.g., dimethylformamide (DMF), dimethylacetamide (DMA), N-methylpyrrolidone (NMP), dimethylsulfoxide (DMSO), dimethylsulfone, dioxane, and diglyme).

There are several problems, however, with the approaches of DE 2718363. For example, the solvents in DE 2718363 work well at low concentrations of salt in solvent (e.g., low concentrations being 0.2 mole of salt in 100 grams of solvent, or a propionic acid/solvent ratio of 0.18). It is, however, desirable to work at higher propionic acid concentrations for an industrial process (e.g., propionic acid concentrations from 18 weight percent (wt. %) to as high as 60 wt. % based on the total weight of the entire solution (e.g., the salt-splitting solution)). Furthermore, DE 2718363 teaches that the use of the lower boiling solvents (e.g., DMF, DMA and DMSO) leads to severe foaming problems that render them unfit for use in a commercial process.

The present disclosure provides a solution to these issues. Surprisingly it has been discovered that the addition of a polar aprotic organic solvent to an aqueous mixture containing an AP, promotes removal of ammonia to a higher degree (higher conversion of salt to ammonia and acid). If an alkanol is employed as the solvent, it forms a heterogeneous azeotropic mixture with the water in the reaction mixture. In the overhead condenser, it is possible to separate the alkanol from the water, and the alkanol can be recycled to the reaction mixture. It has also been surprisingly discovered that the use of the alkanol in the aqueous mixture also mitigates the foaming problems encountered during the heating process (e.g., thermal salt-splitting) with many polar aprotic organic solvents.

As used herein, an “aqueous mixture” can be derived from a fermentation broth or can be the product medium resulting from subjection of microorganisms to one or more fermentation stages in a fermentation medium to produce, among other things, propionic acid and a salt of propionic acid, e.g., ammonium propionate. The fermentation medium, as used herein, means a mixture of water, sugars and dissolved solids that is used in one or more fermentation stages to allow'microorganisms to produce propionic acid and AP, along with other compounds. The aqueous mixture may be subjected to pasteurization, sterilization, purification, filtration, concentration, or a combination thereof. Suitable examples of an aqueous mixture, as used herein, include, but are not limited to, those described in International Publication Number WO 2011/094457, incorporated herein by reference in its entirety.

As used herein, the term “ammonium” (e.g., as in ammonium propionate) refers to a cation having the formula NHR₃⁺ where each R group, independently, is hydrogen or a substituted or unsubstituted alkyl, aryl, aralkyl, or alkoxy group. Preferably, each of the R groups is hydrogen.

As used herein, a “salt-splitting liquid” can have the physical characteristic of being either a mixture or a solution, as are known in the art. The aqueous mixture and the polar aprotic solvent together form the salt-splitting liquid.

As used herein, “thermal salt-splitting” is a process used to “split” or “convert” AP, to propionic acid and ammonia. The thermal salt-splitting process includes heating the salt-splitting liquid that includes the aqueous mixture to convert AP, to propionic acid and ammonia. As such, heating the salt-splitting liquid to convert AP, to propionic acid and ammonia is a thermal salt-splitting process.

The process of the present disclosure converts AP in the aqueous mixture to propionic acid. The process includes admixing a polar aprotic organic solvent with the aqueous mixture, where the ammonium propionate and the propionic acid are soluble in the polar aprotic organic solvent.

The salt-splitting liquid is heated to convert the AP, into propionic acid and ammonia (e.g., TSS). Heating the salt-splitting liquid also produces a vapor phase containing at least water, ammonia and the solvent. At least a portion of the water and the ammonia are removed from the vapor phase during the heating and at least a portion of the solvent is returned from the vapor phase back to the salt-splitting liquid.

As used herein soluble means the ability of AP, and the propionic acid, at the concentrations provided herein, to mix with the polar aprotic organic solvent and the aqueous mixture to form a homogeneous solution (e.g., the aqueous solution of the aqueous mixture). This allows for the solubility of the AP, the propionic acid and the polar aprotic organic solvent in the aqueous mixture (e.g., water, among other compounds). From this homogeneous solution the water and the ammonia can be separated (via the vapor phase), leaving the polar aprotic organic solvent in the salt-splitting liquid. Maintaining the polar aprotic organic solvent in the salt-splitting liquid helps to keep the viscosity of the aqueous mixture low enough (e.g., less than 5000 centiPoise (cP), preferably less than 2000 cP, measured at 25° C.) to allow the salt-splitting liquid to be handled more easily. Maintaining the solvent in the salt-splitting liquid also helps to keep the propionic acid dilute (e.g., a concentration of about 6.3 mole of propionic acid per liter of salt-splitting liquid or less). As such, the present disclosure provides for, among other things, a polar aprotic organic solvent that, when used with an aqueous mixture during heating (e.g., thermal salt-splitting), allow for the solubility of water, propionic acid and the AP in the salt-splitting liquid, the separation of the water and ammonia in the vapor phase, and the polar aprotic organic solvent to remain in the salt-splitting liquid of the aqueous mixture to keep the system viscosity low enough to handle the material and keep the propionic acid dilute enough to minimize side reactions.

Preferably, the polar aprotic organic solvent has a boiling point that is higher than, but close to, that of water (at comparable pressure and temperature). This allows the water to be preferentially removed during the heating process and also facilitates its removal later in the process. As discussed, the water, propionic acid and the AP are all soluble in the polar aprotic solvent (e.g., the polar aprotic solvent is a good solvent for the aqueous mixture). As discussed, the aqueous mixture can be “concentrated” (e.g., where water has been removed from the aqueous mixture prior to the addition of the polar aprotic solvent, such as by rotary evaporation) so as to provide a concentrated aqueous mixture to be used with the present disclosure.

The polar aprotic organic solvent can be one that dissolves both propionic acid and the AP in the required proportions. Examples of the polar aprotic organic solvent include, but are not limited to, those selected from the group consisting of C₄₋₈ alkanols, dimethylformamide (DMF), dimethylacetamide (DMA), N-methylpyrrolidone (NMP), dimethylsulfoxide (DMSO), dimethylsulfone, dioxane, diglyme, or a combination thereof. Examples of C₄₋₈ alkanols include butanol, pentanol, hexanol, heptanol, and octanol. Normal alkanols are preferred.

Determining the amount of polar aprotic organic solvent to admix with the aqueous mixture is based on an assumption that water will be completely removed from the salt-splitting liquid by the end of the process of the present disclosure. The amount of polar aprotic organic solvent to use is, therefore, a function of the desired concentration (e.g., wt. %) of the propionic acid in the polar aprotic organic solvent, as provided herein, at the end of the process. This can also be function of the weight ratio of the propionic acid to the polar aprotic organic solvent.

For the various embodiments the polar aprotic organic solvent is admixed with the aqueous mixture so that a weight ratio of propionic acid to polar aprotic organic solvent in the aqueous mixture is 0.6:1 or lower, preferably 0.18:1 to 0.58:1, more preferably 0.24:1 to 0.42:1. Another approach to how much of the polar aprotic organic solvent is admixed with the aqueous mixture is to have a concentration of up to 60 weight percent (wt. %) of the propionic acid in the salt-splitting liquid, preferably 10 to 40 wt. % of the propionic acid in the salt-splitting liquid, more preferably 11 to 26 wt. % of the propionic acid in the salt-splitting liquid. It is also possible that the propionic acid in the salt-splitting liquid can have a concentration of 15 to 25 wt. % based on the total weight of the salt-splitting liquid. If an alkanol is employed as the solvent, it forms a heterogeneous azeotropic mixture with the water in the reaction mixture. In the overhead condenser, it is possible to separate the alkanol from the water, and the alkanol can be recycled to the reaction mixture. This helps to lower the amount of heat needed during the conversion of the AP to propionic acid by lowering the temperature at which water is removed from the salt-splitting liquid. A result of admixing an azeotroping alkanol is, therefore, to make the water and ammonia removal from the salt-splitting liquid more efficient. In other words, the use of an azeotroping alkanol helps to achieve better removal of both the water and the ammonia from the salt-splitting liquid.

The reaction apparatus used in the process of the present disclosure can include those styled after a Dean-Stark apparatus. Generally, the reaction apparatus includes a heated reactor vessel (e.g., used to heat the salt-splitting liquid and generate the vapor phase discussed herein) having an outlet into a fractionating column structure. The heated reactor can be used to heat the salt-splitting liquid to a temperature of from 80 to 200° C., or from 100° C. to 130° C. The fractionating column structure includes a condenser and a decanter. The vapor phase produced in the heated reactor enters the condenser where it condenses and enters the decanter. In the decanter the condensed salt-splitting liquid is allowed to separate into an aqueous layer and an organic layer. A portion of the aqueous layer (e.g., water and ammonia) is removed while a controlled portion of the organic layer is returned to the salt-splitting liquid in the heated reactor so as to at least maintain the base level of solvent in the salt-splitting liquid. This process ensures that the organic solvent is always present in the salt-splitting liquid during the heating process that converts the ammonium propionate, e.g., AP, to propionic acid and ammonia. The reaction apparatus can be operated at atmospheric pressure, which is preferred. Alternatively, the reaction apparatus can be operated at a reduced pressure (a pressure lower than atmospheric pressure, e.g., under vacuum).

The use of the azeotroping alkanol in the aqueous mixture has also been surprisingly found to control foaming during the heating process (e.g., thermal salt-splitting)

The following examples are provided to illustrate the disclosure, but are not intended to limit the scope thereof.

### Materials

Prepare an aqueous mixture according to Example 11A (the second fermentation) as provided in International Publication Number WO 2011/094457, incorporated herein by reference in its entirety.

All other compounds are purchased from either Sigma/Aldrich or Fisher Scientific and used as supplied.

### Apparatus and Procedure

For the Examples and Comparative Examples listed below the content of the flask are held at reflux temperature, where the temperature varies with the water content in the reaction mixture in the flask. The feed rate of the aqueous mixture is controlled, when needed, to prevent foam from traveling out of the flask and into the Dean-Stark trap.

Express the concentrations of ammonium propionate (AP) as weight percent of propionic acid equivalents (measure by HPLC).

### EXAMPLE 1

### Thermal Salt Splitting of Ammonium Propionate (AP) in the Presence of Polar Aprotic Solvents

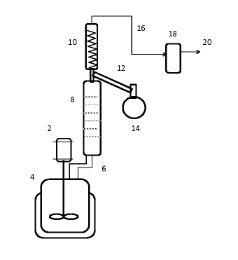

### Description of FIG. 1:

FIG. 1 includes a first vessel 4, which acts as a batch reactor. Vessel 4 is equipped with a hot oil jacket and an agitator 2. FIG. 1 also includes a second vessel 8 that is a distillation column. A third vessel 10 acts as an overhead condenser to the distillation column, a fourth vessel 14 acts as condensate collection pot, and a fifth vessel 18 acts as a cold trap.

Batch reactor 4 is configured to receive an aqueous feed of a composition comprising a mixture of the ammonium salt of PA, protonated PA, and organic solvent. The aqueous feed is an aqueous mixture. The aqueous PA in vessel 4 is maintained at a first temperature under atmospheric pressure. The batch reactor is attached to a small distillation head 8 to bring the heavies down to the batch pot and take lights such as water and ammonia overhead. The distillation head is attached to a total condenser 10 to condense volatiles, such as water and ammonia as ammonium hydroxide, which are collected in condensate collection pot 14. The vent from the condenser goes through a cold trap to the atmosphere.

The clarified aqueous mixture starting material is pre concentrated using a rotary evaporator to remove water to achieve a PA concentration of 20-30 wt. %. Before the pre concentration step in the rotary evaporator, the fermentation broth pH is about 7. The concentrated clarified aqueous mixture pH is 5.5-6.5. During pre-concentration, the rotary evaporator is maintained at 60° C. and 15-60 mbar. Under these conditions, at least 10-20% of the ammonium PA salt is split to produce a mixture of protonated propionic acid and residual ammonium PA salt. This pre-concentrated aqueous mixture is then fed to the batch reactor, to carry out the thermal salt splitting of residual ammonium PA salt.

750 g of a mixture of NMP (N-methyl-2-pyrrolidone) and pre-concentrated aqueous mixture is charged to the reactor. The solvent to pre-concentrated aqueous mixture ratio weight ratio is 4:1. The PA concentration in the pre-concentrated broth is 22 wt. %. Methanol could be added as an optional reagent to push the heavy phase of the water-PA azeotrope back into the reaction mixture. The batch reactor 4 temperature is maintained at 120-125° C. by adjusting the hot oil jacket temperature. Samples are collected from time to time to monitor the progress of the salt splitting and the PA concentration in the reactor. Very little condensation is observed in the overhead condenser 10. However, some amount of liquid is found in the dry ice trap. The total amount of ammonium ion removal is measured by a colorimetric method (Hach's high range (0 to 50 mg/L) ammonia quantification method using a DR 2800 portable spectrometer). The colorimetry method measures the nitrogen associated with NH4+ ion or the NH3 molecule. The PA concentration is measured by HPLC. The results are summarized here:

| | Time | Reactor | Salt | pH of the | PA | % conversion | PA lost in the |
|---|---|---|---|---|---|---|---|
| | (hr:mm) | Temp. C. | splitting yield | reaction | component | to amide | overhead |
| Control | 0:00 | 30 | | 5.68 | 102% | less than | None |
| Sample 1 | 0:30 | 60 | 9.7% | 5.65 | | 1 wt % | |
| Sample 2 | 1:30 | 119.7 | 68.6% | 4.35 | | | |
| Sample 3 | 2:00 | 120.2 | 80.7% | 4.01 | | | |
| Sample 4 | 2:30 | 120.3 | 87.1% | 3.78 | | | |
| Sample 5 | 2:45 | 120.7 | 88.6% | 3.78 | | | |

These results demonstrate the impact of solvent based TSS, where 89% salt splitting yield is achieved without any PA loss in the overhead. The initial PA lost to amide is also minimal in the presence of a solvent.

### Comparative Experiment A

### Not an Embodiment of the Invention

The procedure of Example 1 is repeated without any solvent. Thus, a solventless batch scale study is conducted to evaluate the salt splitting yield and the corresponding generation of amide during the salt splitting and the loss of PA in the overhead. Although a high salt splitting efficiency of more than 80% is achieved, 10+wt. % of initial PA is found in the overhead distillate and 4.8 wt. % of PA is lost as amide.

### EXAMPLE 2

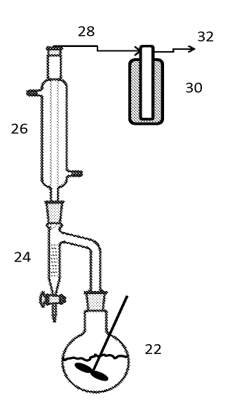

FIG. 2 includes a first vessel 22 which acts as a batch reactor. Vessel 22 is equipped with a hot oil jacket and an agitator. FIG. 2 also includes a second vessel 24 that is a Dean Stark trap, a third vessel 26 that acts as an overhead condenser, and a fourth vessel 30 that acts as the cold trap.

Batch reactor 22 is configured to receive an aqueous feed of composition comprising an aqueous mixture of the ammonium salt of PA, protonated PA and organic solvent. The aqueous mixture is fed to the reactor. The aqueous PA in vessel 22 is maintained at a first temperature under atmospheric pressure. The batch reactor is attached to a Dean Stark trap 24. The Dean Stark trap is used to phase separate the condensed alcohol and water mixture. The Dean Stark trap is connected to an overhead condenser. The vent gas from the overhead condenser passes through cold trap to the atmosphere.

The fermentation broth pre-concentration method of Example 1 is employed. The pre-concentrated broth is then fed to the batch reactor, to carry out the thermal salt splitting of residual ammonium PA salt.

Approximately 100 g of pre-concentrated fermentation broth containing 20-25 wt. % propionic acid as ammonium propionate is mixed with 150 g of n-butanol or n-pentanol or n-hexanol. The reactor is maintained at 100-120° C. by adjusting the hot oil jacket temperature. All the selected alcohols (C4-C6) form a heterogeneous azeotrope with water. A Dean Stark trap attached to the overhead condenser is used to separate the alcohol and water phases upon condensation. The water layer is discarded and the alcohol phase is continuously returned back to the reactor. The reaction is continued at the desired temperature (100-120° C.) for 1-2 hrs. The initial feed sample, the final reactor sample, the distillate water phase and the alcohol phase trapped in the dean stark trap are analyzed to determine the propionic acid mass balance and the salt splitting yield. The results are summarized here:

| | n-butanol | n-pentanol | n-hexanol |
|---|---|---|---|
| Alcohol to aqueous mixture | 1.5 | 1.5 | 1.5 |
| wt. ratio | | | |
| Feed PA conc., by wt. | 8.23% | 8.02% | 8.20% |
| Final PA concentration, by | 9.60% | 8.30% | 8.50% |
| wt. | | | |
| Final ester conc., by wt. | 4.70% | 5.40% | 4.90% |
| Mol balance accountability | 97% | 94% | 101% |
| Conversion to ester | 20% | 25% | 25% |
| Initial water conc. in the feed, | 46.3% | 46.3% | 46.3% |
| wt. % | | | |
| Final water conc., wt. % | 2.5% | 3.4% | 3.5% |
| Salt splitting yield | >70-75%< | >90% | >70-75%< |
| PA loss in the form of amide | 1.30% | 1.80% | 0.60% |
| PA loss in the distillate | <1 wt. % | <1 wt. % | <1 wt. % |
| (Water or alcohol phase) | | | |

PA loss in the overhead and the conversion to amide are minimal compared with the solventless thermal salt splitting experiments in Comparative Experiment A. Around 20-25% of the initial PA is converted to ester in the presence of the alcohol.

1. A process of converting an ammonium propionate in an aqueous mixture to propionic acid, the process comprising:
admixing a polar aprotic organic solvent and the aqueous mixture, where the ammonium propionate and the propionic acid are at least partially soluble in the polar aprotic organic solvent, to form a salt-splitting liquid;
heating the salt-splitting liquid to convert the ammonium propionate to propionic acid and ammonia, where heating the salt-splitting liquid produces a vapor phase containing at least water, ammonia and the solvent;
removing at least a portion of the water and the ammonia from the vapor phase during the heating; and
returning at least a portion of the solvent from the vapor phase back to the salt-splitting liquid.

2. The process of claim 1 including heating the salt-splitting liquid to a temperature of 80° C. to 200° C.

3. The process of claim 1, including heating the salt-splitting liquid to a temperature of 100° C. to 130° C.

4. The process of claim 1, where the polar aprotic organic solvent is selected from the group consisting of C₄₋₈ alkanols, dimethylformamide (DMF), dimethylacetamide (DMA), N-methyl-2-pyrrolidone (NMP), dimethyl sulfoxide (DMSO), dimethylsulfone, dioxane, diglyme, or a combination thereof.

5. The process of claim 1, where the polar aprotic organic solvent is a C₄₋₈ alkanol.

6. The process of claim 1, where the alkanol is selected from the group consisting of butanol, pentanol, hexanol, heptanol, and octanol.

7. The process of claim 1, where the alkanol is selected from the group consisting of n-butanol, and n-pentanol.

8. The process of claim 1, where a weight ratio of propionic acid to polar aprotic organic solvent in the aqueous mixture is 0.6:1 or lower.

9. The process of claim 1, where a weight ratio of propionic acid to polar aprotic organic solvent in the aqueous mixture is 0.18:1 to 0.58:1.

10. The process of claim 1, where the polar aprotic organic solvent is N-methyl-2-pyrrolidone.

11. The process of claim 1, where at least one C₄₋₈ alkanol is employed as an azeotroping solvent.

12. The process of claim 1 wherein the conversion of propionic acid to amide is less than 2 wt. % preferably less than 1 wt. %.

13. The process of claim 1 wherein the loss of propionic acid in the overhead is less than 1 wt. % based on the initial propionic acid content of the aqueous mixture.
